(19) United States
(12) Patent Application Publication
(10) Pub. No.: US 2002/0063850 A1
(43) Pub. Date: May 30, 2002

(54) Method of selection of eye position data and apparatus for carrying out the method

(76) Inventors: Jean-Cyriaque Barry, Tubingen (DE); Stefan Rensing, Aachen (DE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/866,250

(22) Filed: May 25, 2001

Related U.S. Application Data
(63) Continuation of application No. PCT/EP99/09279, filed on Nov. 29, 1999.

(30) Foreign Application Priority Data
Nov. 27, 1998 (DE) 19854852.4

(51) Int. Cl. A61B 3/14

(52) U.S. Cl. 351/209

(57) ABSTRACT

The invention relates to a method of selection of eye position data of a test person suitable for further processing from at least one data series associated with at least one eye. The data is supplied by an eye position measuring instrument within a certain time interval, where the data is initially stored. Subsequently, the individual data points of the data series lying within a predetermined window interval are collected into clusters. Each cluster is then assigned to a group, where the first group contains the cluster with the first data point in time and the further clusters are assigned to further groups corresponding to the time sequence of the date points. Finally, those groups suitable for further processing are selected which have the most data points. The invention also relates to an apparatus for carrying out the present method.

### RELATED APPLICATIONS

This application is a continuation of PCT-application PCT/EP99/0279, filed Nov. 29, 1999 and claiming priority of application DE 19854852, filed Nov. 27, 1998.

### BACKGROUND OF THE INVENTION

1. Field of the Invention

The invention relates to a method for selection of eye position data from a test person suitable for further processing out of a series of data associated with at least one eye, which are taken from an eye position measurement instrument within a given time interval. The invention further relates to an apparatus for selection of eye position data, comprising an eye position measuring instrument which provides data as a series of measurement data from at least one eye within a predetermined time interval and a data analysis device.

2. Description of the Related Art

The position of the eye is measured in the field of behavioral psychology, in science, in animal experiments and in many other technical fields. Various methods and apparatus for detecting eye movement are disclosed in the documents DE 19 624 135 A1, U.S. Pat. No. 4,859,050 A or DE 44 08 858 A1. In several of the known methods, it is necessary to first perform an individual calibration, which requires cooperation of the test person to the extent that they must precisely view a calibration object for a sufficiently long time when asked.

In ophthalmic diagnostics, objective eye position investigations are carried out, which can lead to early detection of a squinting or a strabismal condition. The basis of such investigations is eye position data, which are measurable objectively, i.e. without information about the person or patient being studied, in various methods, such as the Purkinje reflect pattern method. The eye position data taken must then be evaluated by the investigator. The data includes a horizontal and a vertical angular position for each eye.

The investigator must determine the interrelationship of these data to a fixation point, which the patient has viewed during the investigation. Generally, the investigator is not in the position to decide based on the collected data whether the patient actually fixed his view to the fixation point, The analysis of the data often leads to incorrect results, especially for children, retarded persons, animals, etc, who do not understand or do not follow the instructions of the investigator, such that the investigator possibly assumes a fixation point which was not actually fixed.

In addition, the eye position data itself is subject to errors. A possible source is the methodic inaccuracy of the eye position measurement, where here a systematic error (off-set) and a nonsystematic error (noise) are mentioned. A second error source lies in the lack of fixation capability, which depends on the patient and the age of the patient. It will be understood that the capability of fixation depends on age within a certain range, whereby this fluctuation range becomes smaller with visual maturity and can increase due to illness or age,

A further error source results from an individual off-set between the eye position and the actual view direction, which is different for the left and right eye, depending on the parameter measured. For example when measuring the eye position by means of cornea reflex, there is an angle between the visual axis or view axis and the axis normal to the apex of the cornea, in optometry the so-called kappa angle. This angle is different in different individuals and is normally not exactly the same in the left and right eyes.

Finally, errors can also arise because the patient does not correctly fix monocularly and binocularly.

Consequently, eye position data are more or less strongly subject to errors or defects depending on the patient and the measurement method.

Based on the eye position data, the investigator has the task of determining in which direction the patient was viewing, or whether he was properly fixing monocularly (i.e. squinting), or whether he was properly fixing binocularly, i.e. he was simply viewing in another direction (false fixation). Thus it is necessary that the investigator establish a relation between the eye position data and the point to be fixed by the patient. If the differences in the data over time are much larger than the measurement accuracy and the fixation capability, it is not difficult for the investigator to sort the eye position date based on large thresholds.

It becomes a problem however if the measurement accuracy and/or the fixation capability or the false fixations are on the same order of the measured eye position and for example when an analysis is made online with video frequency. A human investigator cannot exactly, objectively, reproducibly and rapidly accomplish this. In addition, the necessary analysis of the eye position data can only be undertaken by highly qualified persons, so that early recognition in the scope of preventive tests in children by non-specialized physicians is not possibe.

In view of the above, the object of the invention is to provide a method and an apparatus which allow an economic, objective, reproducible test and analysis of the eye position independent of the investigator. In particular, the method should be such that it can be carried out by persons not having special training. Further, the method and the apparatus should select the data indicating a false fixation out of the measured eye position data.

### SUMMARY OF THE INVENTION

The object underlying the invention is achieved in a method for selection of eye position data of a test person, which comprises the following steps:

storing the measured data,

collecting the individual data points of the data series lying within a predetermined window interval into clusters,

assigning each cluster to a group, where the first group conmprises the cluster with the first data point in time and the further clusters ate assigned to further groups corresponding to the time sequence of the data, and

selecting those groups having the most data points to be suitable for further processing.

This method has the advantage that the data indicating a false fixation are marked as being not suited for further processing and are therefore separated out. Thus it is possible with the present method to determine those eye position data, which indicate with high probability that the test person has fixed on the given point at least with one eye. These data can then be readily processed further in corresponding computational algorithms, such that a first result can be established without the efforts of an skilled investigators.

A further advantage of the present method can be seen in that the test person need not fix onto a predetermined fixation point. It is sufficient for an eye position test to carry out the measurement over a certain time, where the present method selects only those data which indicate with high probability that a fixation was made to an arbitrary point in space. This can be used for self-calibration of devices for a given test person, so that even uncooperative test persons or animals, through repeated uninstructed viewing of predetermined calibration objects without coercion, finally deliver the necessary data for interrelating a measurement signal of a certain strength and configuration to the given objects, because generally with time any distinctive structure will be viewed more frequently and more precisely than a plain background. The considerable advantage of the present method therefore is that an investigation of the eye position, the fixation, and possible optometric anomalies of the patient is possible on an objective basis.

In an advantageous embodiment of the present method, the series of data associated with one eye consist of data pairs, where each data pair represents the horizontal and vertical angle of the eye position. Preferably, a series of data is taken for each eye and stored in memory. Preferably, the collection into clusters is carried out for the individual data points of the data pairs. Preferably, the time sequence is measured.

The use of horizontal and vertical angles to describe the eye position as well as the detection of a data series for each eye has proven to be particularly advantageous. It will be understood that other coordinate and reference systems are possible, for example polar coordinates or vector representations.

In a preferred embodiment of the present method, the window range is selected to depend on the age of the test person. Namely, it has been shown that fixation capability depends on age, where older children and adults are better able to fix an object over a given time period than for example babies or small children or patients or animals with eye disorders.

The advantage is that this age dependent distinction in fixation capability is included in the method, so that the selection of the eye position data in the end can be more reliable.

In a preferred embodiment of the present method the selected data for the left and right eye are compared to one another, where a deviation exceeding a certain amount indicates squinting of the test person. This has the advantage that an initial finding is automatically possible without the efforts of a person specialized in eye testing.

The object underlying the invention is also achieved with an apparatus of the mentioned type, which is characterized in that the data analysis means comprises a memory as well as means for collecting data into clusters, means for ordering clusters to groups and means for selecting a group of data suitable for further processing.

This apparatus suited for carrying out the inventive method has the advantage that an investigation of eye position anomalies is also possible by unskilled persons on objective basis. In particular, the present apparatus has the advantage that data indicative of a false fixation can be recognized and for example be separated out.

In a further preferred embodiment of the present apparatus, the means for collecting data is configured such that it collects individual data points of the data series lying within at least one predetermined window range or interval, where the largest data point is assigned to the first cluster and the smallest data point to the last cluster. Preferably, the means for ordering clusters subdivides them into groups, where the cluster with the first data point in time is assigned to the first group and the further clusters are assigned to further groups corresponding to the further time sequence of the data points. Preferably, the means for selection selects the group having the most data points. An apparatus configured in this manner has proven to be particularly advantageous with respect to the quality of data selection.

In a further embodiment of the invention, the eye position measuring instrument comprises an infrared light source directed to the eyes and a video camera for recording the eyes. The use of an infrared light source has the advantage that the test person is not disturbed or irritated by a blinding light, because infrared light is hardly visible for the test person.

For example, with the aid of the so-called Purkinje reflex pattern method, the images of the eye recorded by the video camera can be analyzed and the eye position angle relative to the straight ahead position can be calculated. It will be understood that other methods can also be used to determine the eye position data, for example electro-oculargraphy, search coil methods, foveal birefringence (FB) scanning, cornea reflex measurement, infrared reflection, shifting of eye structures (e.g. limbus corneae, pupils) by means of CCD lines or image processing, dual Purkinje image eye tracking, determining the iris torsion, power retractor according to Weiss and Schaeffel (University of Tübingen) and OVAS system (Ocular Vergence and Accommodation System). The method according to the present invention is not limited to the eye position data detected by these special methods. Further advantages and embodiments of the invention result from the description and the attached drawings.

It will be understood that the above-mentioned features and those to be discussed below are not only applicable in the given combinations, but may be used in other combinations or taken alone without departing from the scope of the present invention.

The invention will now be described in more detail in terms of embodiments taken in conjunction with the drawings.

### BRIEF DESCRIPTION OF THE DRAWINGS

FIG. 1a shows a table with various eye position data.

FIG. 1b shows a diagram for explaining the collection into clusters based on the data given in FIG. 1a.

FIG. 1c shows an example of the age dependency of fixation accuracy when using a handheld device for early detection of faulty eye positions.

FIGS. 2a-d show four tables with measured data pairs for the left and right eyes for illustration of the present method.

FIGS. 3a-d show four tables of measured data pairs for the left and right eyes according to a second embodiment for illustration of the present method.

FIG. 4a shows a schematic illustration of a present apparatus for selection of eye position data.

FIG. 4b shows a schematic block diagram of a data analysis device according to the present invention.

### DESCRIPTION OF THE PREFERRED EMBODIMENTS

The present method is described in the following in an example for investigating eye position anomalies (squinting). It will be understood that the present method as well as the present apparatus can also be employed in other applications. It is contemplated for example to employ the present method to observe persons looking into a shopping window and to determine which object or region within the shopping window is observed most frequently and for the longest time. Valuable conclusions can then be made for example as to which product receives the most attention or which area of the shopping window is best suited for the presentation of goods.

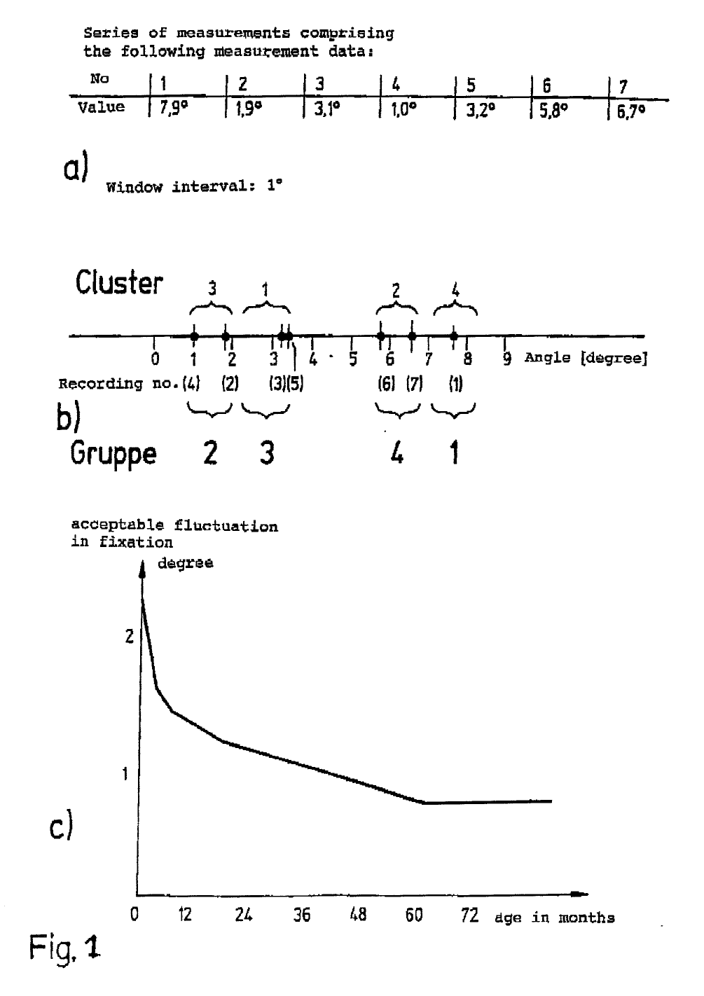

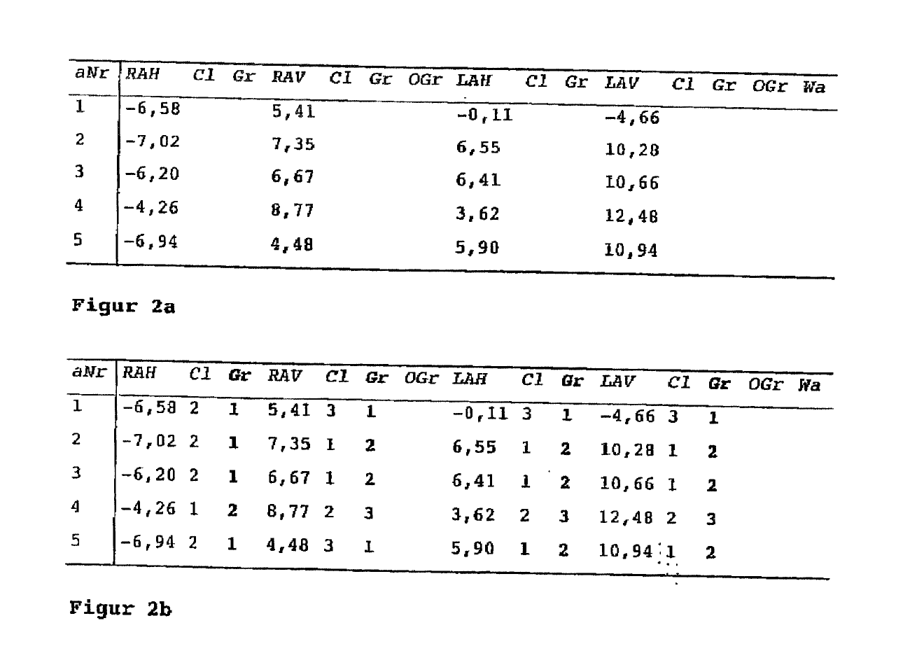

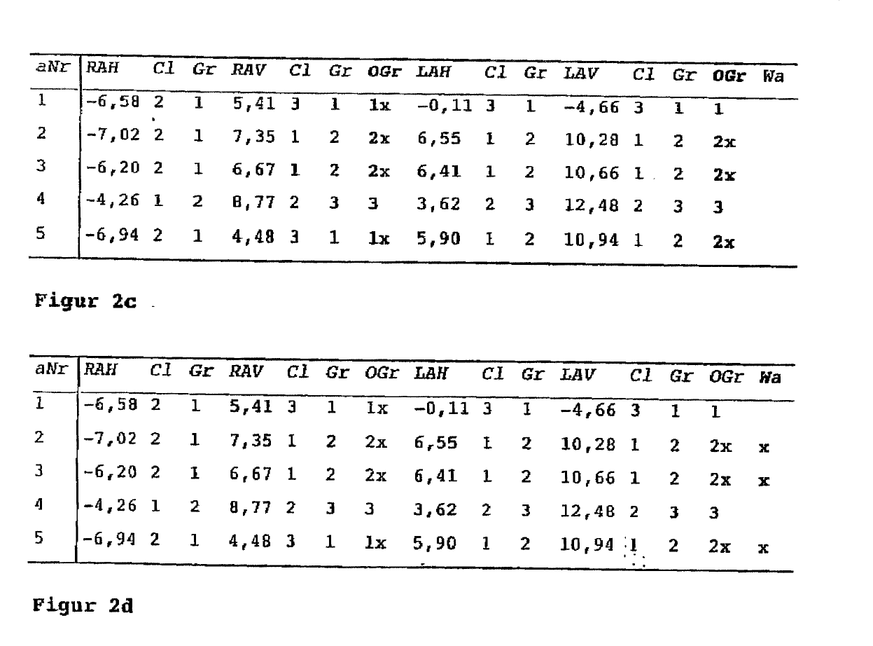

An eye position measuring instrument to be described later provides a plurality of data points over a given time interval, for example ten minutes. The data are arranged in two data series, where each data series contains the horizontal and vertical eye position angle of the left and respectively the right eye. The eye position angle is measured relative to one eye position, for example the straight ahead view. FIG. 1 shows measured data limited to one spatial direction for the vertical eye position of one eye. The first data point is 7.9°, the following data points in time sequence are 1.9°, 3.1°, 1.0°, 3.2°, 5.8° and 6.7°. It is clearly recognizable that the data fluctuate very strongly, so that the impression at first arises that the test person has not fixed on only one object during the measuring time interval.

To further process the data, the data points indicative of a false fixation must be separated out. For this purpose, the data is ordered in so-called clusters. One cluster represents a data range of a certain width, in which the most possible individual data points fall, which occur within a certain time interval. The width of this data range is given by the diagram in FIG. 1c, in which the allowable fixation fluctuation for the respective eye position measuring method is given in degrees as a function of the age of the test person in months. This diagram accounts for the fact that the fixation capability, i.e. the capability to maintain fixation as far as possible without fluctuation, becomes better with increasing age. For example, for a baby, the fixation fluctuation for this eye position measurement method is in the range of 2.5°, while the fixation fluctuation for a grown person decreases to a few tenths of a degree. This has the consequence with respect to data analysis, that data for a small child lying within the region of 1.5 to 2.5° are indeed indicative of a fixation on a certain point, while such a variation for a grown person would be considered to be faulty fixation.

The data given in FIG. 1a is plotted as a data string in FIG. 1b. With this data string, it is then determined which data ranges can be formed with respectively the maximum number of data points. The width of the data range is determined from the diagram in FIG. 1c based on the age of the test person. It will be understood that other factors can also be accounted for in these data ranges, for example factors depending on the eye position measurement method used. In additions it is also possible to use different data ranges for the left and right eye or for the vertical and horizontal directions.

In the present embodiment, a fixation fluctuation or width of 1° is used, in the following also referred to as the window interval. The aim is to determine whether a test person has viewed a fixation point or not with one or both eyes.

A sorting algorithm is used for the actual determination of which data is to be associated with a certain view position. The algorithm is flexible and can be adapted to the purpose of the analysis. In this example, the analysis of 4 dimensional data sets (generally: n-dimensional) is concerned, so that the method includes 4 (n) steps, where the final selection takes place in the last step. The steps are carried out in a certain sequence:

1. Cluster formation into stages to determine which one dimensional data of one eye (horizontal or vertical view direction) can be maximally arranged within a certain window interval,

2. Group formation for arrangement according to time sequence and duration,

3. Higher group formation to determine which groups of respective right and left eyes are associated with one another,

4. Ordering of the higher groups of the left and right eyes to determine which groups of both eyes are associated with one another.

The final selection of the data then follows depending on the application.

Stage 1 of cluster formation: the data are arranged according to size in decreasing sequence to determine the clusters as shown in FIG. 1b. The number of elements and density, e.g. its distance squared summation, is determined for each cluster. The first cluster from the right begins with the largest value 7.9° and contains only one element in this case and therefore has the distance squared summation of 0, because the distance to the next data point is more than 1°.

The next cluster begins with the second largest element, contains two values (6.7° and 5.8° ) and the distance squared summation is 0.81°. This continues from the right to the last cluster, which here then contains only the last and the smallest value.

Stage 2 of cluster formation: now the clusters are numbered according to the number of elements, so that all elements of the cluster have the same cluster number, namely the cluster with the most elements has the number “1”, the cluster with the second largest number of elements has the number “2” and this continues in decreasing sequence of the number of elements. To provide uniqueness, each number is only used once. Should several clusters have the same number of elements, so that their intersecting set is non-zero, then the next criterion is considered, for example the magnitude of the distance squared summation, to decide which values are to be marked as belonging to the cluster. The smallest summation has the highest ranking, i.e. leads to the next cluster formation, etc. Further criteria, random mechanisms and case distinctions can be employed to ensure the uniqueness of the ordering.

At the end of cluster formation, see FIG. 1b, each value is uniquely assigned to a cluster, so that always the most possible number of elements is contained in one cluster, where the predetermined window interval is maintained. If it is desired to account for the time sequence of the measurements, it can be required that only those values be assigned to a cluster which follow one another directly and the duration of uninterrupted fixation on a certain location can be derived therefrom. Further time dependent selection criteria can be applied.

In the present case, the time dimension is not to be considered further. As illustrated in FIG. 1b, four clusters exist, three having two values and one having one value. Cluster 1 contains the data numbers 3 and 5 and the cluster elements are particularly close to one another because it has the smallest distance squared summation. The two other oldsters with the same number of elements have the sate distance squared summation, namely 0.81°. In this case, the cluster containing the largest value is given the higher ranking. The fourth cluster is the one with the smallest number of elements, in this case, one element.

The clusters therefore contain those data which lie with the allowable range of fixation fluctuation and thus indicate with high probability that the eye has fixed upon an object when taking these data.

After forming the clusters the individual clusters are ordered in so-called groups, in the case that a time dependent evaluation is desired. While the sequencing of the clusters accounts for the number of data contained therein, the ordering in groups is to account for the time sequence of the recorded data.

In the present embodiment, the data point 7.9° was taken first. The data points 1.9°, 3.1°, 1.0°, 3.2°, 5.8° and 6.7° then followed. The recording number 1 is associated with the group 1. All elements belonging to the same cluster are also assigned to this group (here only the element 1 from cluster 4). With this, the values in this group are removed from the further arrangement in groups. The smallest recording number, which has not yet been allotted, determines the allotment to the following group 2, in this case the recording numbers 2 and 4 from cluster 3. The next group 3 includes the recording numbers 3 and 5 from cluster 1. The remaining recording numbers 6 and 7 form group 4 out of cluster 2.

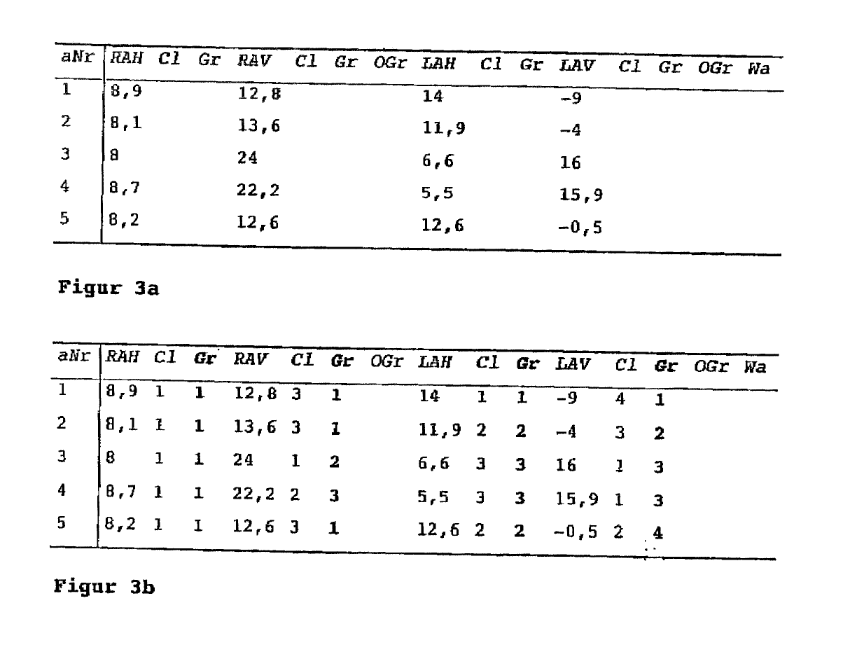

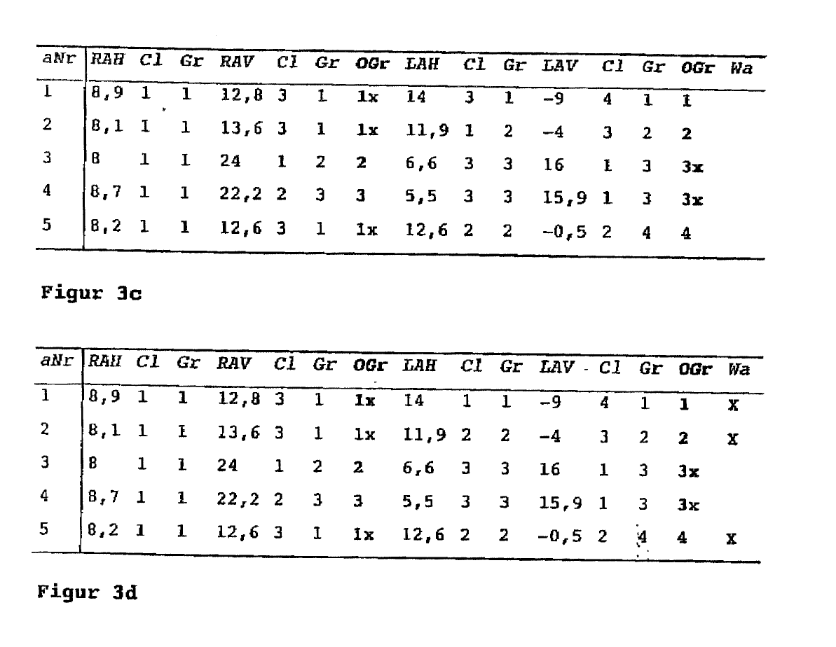

Up until now, only one parameter of one eye has been considered, e.g. the horizontal eye position. The decision is now made on the basis of this group formation as to which data are to be marked for further processing, i.e. which data indicate with high probability that the test person had fixed on an object. This selection will now be explained in conjunction with the tables in FIGS. 2 and 3. FIG. 2a shows a table in which four data series are arranged in columns (horizontal and vertical eye positions) of the right (RAH and RAV) and the left eye (LAH and LAV)). The values of five measurements in time sequence are given in 5 lines.

The same cluster and group formation is carried out for each of the four corresponding series of data (columns). In this example, a window interval of 1.75° is assumed. With this, the ordering of the data is made as shown in the table of FIG. 2b.

A higher group formation now follows separately for each eye. Each horizontal and vertical group number combination forms a higher group. The following results:

Recording number 1, right eye; group 1 & group 1 result in higher group 1; recording number 2, right eye: 1 & 2 result in higher group 2; recording number 3, right eye: the same higher group as recording number 2, right eye; recording number 4, right eye: 2 & 3 result in higher group 3; recording number 5, right eye: the same higher group as recording number 1, right eye.

The same procedure is then followed for the data of the left eye.

The higher groups represent horizontal and vertical pairs of values falling in a common window interval. In this example, this is the value pairs which represent the same horizontal and vertical eye position of an eye. The results of this higher group formation are collected in FIG. 2c.

Now the final selection is made on the basis of the higher group assignments. The relevant criterion in this application is the largest number of value pairs in a higher group. In this example, 2 largest higher groups are present for the right eye with 2 value pairs each and for the left eye 1 largest higher group with 3 value pairs.

All value pairs (recording numbers) belonging to a maximal higher group of one eye are marked as belonging to a maximal higher group, independent of whether only one maximal or several equally large groups exist. This marking is illustrated by an “x” in FIG. 2c.

This has the consequence that the value pair 4 of the right eye and the value pairs 1 and 4 of the left eye are seen to represent false fixations (i.e. the condition that at least one eye fixes on the object is not fulfilled), because they are not contained in one of the largest higher groups.

The number of data pairs included in each higher group is considered for the final selection of the value pairs, here for determining the associated eye positions. The more frequently similar value pairs occur, the higher the probability that a certain object was continuously or repeatedly fixed, This selection criterion is used in this embodiment, although it need not always be used for selecting the associated data sets of the two eyes.

The largest higher group of the right eye is compared to the largest higher group of the left eye. This independent of whether several equally large higher groups are present for one eye. If one of the two groups has a larger number of elements than the other, the lines of the data table belonging to this higher group is finally taken for further processing.

Should the maximal higher groups of the two eyes contain the same number of elements, the lines are initially selected in which both the value pair of the right eye and the value pair of the left eye are commonly marked as belonging to one higher group. If fewer common data lines are present then the maximal number of elements in a largest higher group, then those additional lines are selected in which only one value pair is marked as belonging to the largest higher group. As an arbitrary criterion, the process begins with the right eye.

In this embodiment, maximally 5 data lines (recordings) are to be selected. The result of the final selection is that the recording numbers 2, 3 and 5 are marked for further processing, which is illustrated in FIG. 2d in the column selection (Wa) with an “x”.

One can determine a minimal number of data lines which must be present such that the eye position determination is reliable at all, in the present embodiment this is 3 data lines (recordings and the associated 4-dimensional data sets).

The analyzable data recordings from a test comprise the selected data for the two eyes. Case distinctions are made in the higher group selection and in the final selection to achieve certain analysis goals and to account for special situations, e.g. when a one-sided or alternating fixation occurs or when eye twitching of a squinting patient occurs or when other limiting conditions arise.

The data selected according to this scheme can now be supplied to a squint evaluation. A squint evaluation of the data can be carried out independently of the person running the eye tests, for example with the aid of the so-called strabismus index method. A description of this method can be found in the publication “Statistical Validation of a Strabismus Index Calculated from Objective Ocular Alignment Data”, J. C. Barry et al., Strabismus 1996, Volume 4, No. 2, pages 57-68, whose disclosure with respect to the described method is embodied herein.

The preferably computer-supported analysis of the data in FIG. 2 using the strabismus index method leads to usual results, so that it can be assumed that the test person does not squint and has fixed onto a given point.

FIGS. 3a-d illustrate tables corresponding to the format used in FIG. 2 and containing data of a second test person as an example. As described above, the collection of the data into clusters is undertaken initially, which in a next step are then allotted to groups. As can be clearly seen, the horizontal data of the right eye (RAH) fall within the window interval of one degree and thus are all assigned to cluster 1. In contrast, the vertical data of the left eye (LAV) fluctuate to a great extent, so that the data on the whole is assigned to four clusters.

A comparison of the resulting higher groups with respect to the left and the right eye show that the group with the most data points is group 1 for the right eye and group 3 for the left eye. The comparison of group 1 of the right eye and group 3 of the left eye with respect to the number of data points shows that group 1 of the right eye has the most data and consequently is to be selected for further processing and analysis. Therefore, the data with the numbers 3 and 4 are separated out, while the data indicated with “x” in FIG. 3c and d with the numbers 1, 2 and 5 are passed on to further data analysis.

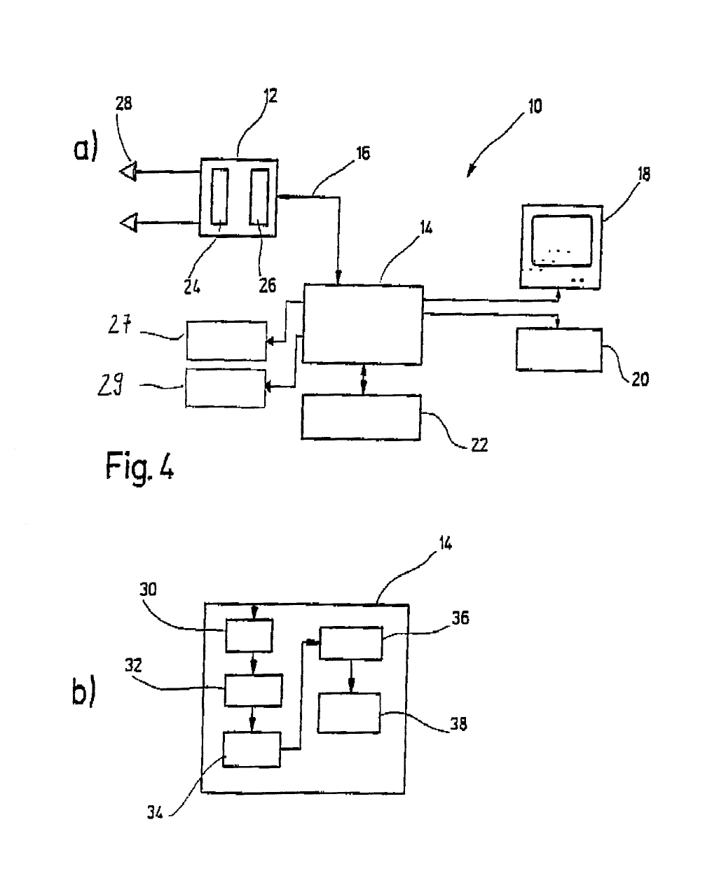

With the aid of the strabismus index method, it results from these selected data that the test person squints with high probability, so that further testing by an ophthalmologist appears to be necessary. The advantage of the above-described method is therefore, among others, that the data can be selected and passed on to analysis without effort of the person performing the test. A corresponding apparatus for carrying out this method will now be described with reference to FIG. 4.

The measuring apparatus is designated with the numeral 10 in FIG. 4a. It includes an eye position measuring instrument 12 connected to a selection and analysis device 14 through a data line 16. The selection and analysis device 14 in turn is connected through corresponding data lines to a monitor 18, a printer 20 and an operating field, for example a keyboard 22. The keyboard 22 serves to input data, for example the personal data of the test person, while the monitor is and the printer 20 provide a representation of the measured data and the analysis results.

The eye position measuring instrument 12 preferably comprises an infrared light source 24 and a video camera 26. The IR light source 24 is provided to irradiate the eyes 26 of a test person. The video camera 26 is directed to both eyes. 28 to make recordings. The eye position of the test person is determined from the video recording with known methods.

The selection and analysis device 14 comprises a memory 30 for storing the measured data for selection and analysis of the eye position data in the above-described method. A device 32 for collecting the data in clusters accesses the memory 30, An ordering device 34 is connected to the device 32, which allots the clusters to certain groups. The determined groups are classified in “fixing” and “non-fixing” groups in a selection device 36. The data classified as “fixing” are supplied to an analysis device 38 from the selection device 36, which analyzes the data and transmits the results for optical display to the monitor 18 and/or to the printer 20. As seen in FIG. 4b, the individual devices 30-38 are connected to one another through individual data lines. It will be understood that a connection of the individual devices can also take place over a common bus line.

In a particularly preferred embodiment, the selection and analysis device 14 is part of a computer. In a further advantageous embodiment, the measuring apparatus 10 comprises an optical stimulator 27, preferably a lamp, and a fixation enhancement means 29 (abbreviated FU means), preferably in the form a melody reproduction device. Both the stimulator 27 and the FU means 29 are connected to the selection and analysis device 14 through data lines and are controlled by same.

At the beginning of the test/measurement, the optical stimulator 28 serves to direct the view of the test person, preferably by a blinking lamp, to a certain point to thereby determine reference or calibration data indicative of a fixation.

Especially with small children, the view often departs rapidly away from such a stimulator, so that normally only a few data are present representing a fixation, which makes the data analysis for diagnosis difficult.

The test persons are subjected to different optical stimuli (fixation objects) during the investigation to achieve a positive enhancement for supporting fixation on a given point. At the same time, the eye position data are analyzed and compared to the calibration data. If the selection and the analysis device 14 determines that fixation on the object is present, i.e. that the test person spontaneously observes the fixation object, it then activates the FU means 29, which plays a melody in response or “rewards” the test person in some other way.

The positive reinforcement during the test, especially for small children has the result that a larger number of data are available from which one can conclude that a fixation on a certain point was present.

It has been found that an automatic detection and analysis of eye position data of a test person is possible with the described apparatus 10, so that for example a test for squinting or strabismus can be carried out by a person who is not an ophthalmologist, without diminishing the quality of the test results.

As already mentioned, the present method and the present apparatus are not limited to applications in the medical field. The apparatus 10 can also be used for example to determine which points are frequently fixed by a person within a certain time interval. A self-calibration of the eye position measuring method is also possible, which presumes the fixation on certain points by the test person before the measurement. This can be used for example for individual calibration or the viewfinder in a camera, which measure the view direction of the person being photographed or whose video image is being taken and automatically focus on the corresponding parts of the image.

The present method and the present apparatus can also be used when the eye position data are provided as a series of individual measurements of the eye position, up to a quasi-continuous measurement or a real time measurement. Examples are the control of the proper eye position in the perimeter (view field tests) or in photo-refractive leaer surgery of the cornea, e.g. for correction of detraction errors. The eye position data produced through fixation of a moving object can also be further processed in the present method and apparatus. In this case, the measured eye position data are calculated in comparison to the known space-time trajectory of the object and the deviation from the desired position is subjected to the selection method according to the present invention. The optical depth or fixation plane can also be determined through the convergence of the eye position, if one allows fixation not only in one plane, but in several planes.

The description relates to embodiments in which the horizontal and vertical eye position components were selected. It will be understood that the present method can also be used for data indicative of the rotation of the eye about the position axis (cyclorotational) and/or the accommodation depth (focal plane of the eye), or further measurement parameters related to the eye position. It is only necessary that the corresponding window intervals be employed.

It will be understood that the present method can be employed not only for the selection of eye position data, but also generally for the selection of arbitrary data of a series of measurement data.

What is claimed is:

1. A method for selecting eye position data of a test subject suitable for further processing from a series of individual data points associated with an eye, said data points being provided by an eye position measuring instrument within a certain time interval, the method comprising:
storing the individual data points;
collecting the individual data points that lie within a predetermined window interval into clusters;
assigning each cluster to a group, wherein a first group comprises the cluster with a first data point in time, and further clusters are assigned to further groups corresponding to a time sequence of the data points; and
selecting those groups having the most data points to be suitable for further processing.

2. The method of claim 1, wherein the series of data points associated with one eye comprises data pairs, wherein each data pair indicates a horizontal and a vertical eye position angle.

3. The method of claim 1, wherein a series of data points is stored for each eye.

4. The method of claim 3, wherein collecting of data into clusters is carried out for the individual data points of the data pairs.

5. The method of claim 2, wherein the collecting of data into clusters and the assigning of clusters to groups is carried out for each data point of a data pair, such that a group pair results for each data pair.

6. The method of claim 5, further comprising allotting the data pairs to higher groups comprising data pairs that fall within a common window interval, wherein the higher groups contain the data pairs that are assigned to the same group pair.

7. The method of claim 1, wherein the window interval is selected depending on the age of the test person.

8. The method of claim 1, further comprising comparing selected eye position data obtained for both left and right eyes, and wherein a deviation exceeding a certain value is indicative of squinting of the test person.

9. Method of claim 1, wherein the individual data points that lie within the window interval follow one another sequentially in time.

10. An apparatus for selection of eye position data, comprising:
an eye position measuring instrument which provides a series of data within a predetermined time interval for at least one eye; and
a data analysis device, comprising a memory, a means for collecting the data into clusters, a means for assigning the clusters to groups, and a means for selecting a group of data as being suitable for further processing.

11. The apparatus of claim 10, wherein the means for collecting data into clusters is adapted to collect individual data points that lie within a predetermined window interval into clusters, wherein a largest data point belongs to a first cluster and a smallest data point to a last cluster.

12. The apparatus of claim 10, wherein the means for assigning the clusters to groups is adapted to assign clusters to groups based on a time sequence, wherein a cluster having a first data point in time is assigned to a first group and a cluster having a second data point in time is assigned to a second group.

13. The apparatus of claim 12, wherein the means for assigning the clusters to groups further comprises a means for allotting the groups into higher groups, wherein data pairs of two series are formed and these data pairs are allotted to a higher group, such that one higher group contains respective data pairs belonging to the same group.

14. The apparatus of claim 10, wherein the means for selecting a group of data is adapted to select the groups containing the most data points.

15. The apparatus of claim 10, wherein the eye position measuring instrument further comprises an infrared light source directed to the at least one eye, and a video camera for recording the at least one eye.

16. The apparatus of claim 10, further comprising an optical stimulator for supplying an optical stimulus, and a fixation support device, wherein both the optical stimulator and the fixation support device is controlled by the data analysis device, wherein control of the fixation support device depends on an emission of the optical stimulus and an analysis of the eye position data.

17. A method for self-calibration of an eye position measuring apparatus, comprising the steps of claim 1.

18. A method for selecting data suitable for further processing from a data series provided by a measuring instrument within a time interval, comprising:
storing the data series;
collecting individual data points from the data series which lie within a predetermined window interval into clusters;
assigning each cluster to a group, wherein the first group contains the cluster with the first data point in time and the further clusters are assigned to further groups corresponding to a time sequence of the data points; and
selecting those groups which contain the most data points as being suitable for further processing.
